(19) United States
(12) Patent Application Publication
(10) Pub. No.: US 2014/0083878 A1
(43) Pub. Date: Mar. 27, 2014

(54) TRANSDERMAL DRUG DELIVERY DEVICE

(71) Applicant: MYLAN INC., Morgantown, WV (US)

(72) Inventors: Jiansheng TANG, Morgantown, WV (US); Shenshen CAI, Morgantown, WV (US); Bhushan KATKADE, Morgantown, WV (US); William C. SCHUMACHER, Morgantown, WV (US); Kenneth J. MILLER, Uniontown, PA (US)

(73) Assignee: MYLAN INC., Morgantown, WV (US)

(21) Appl. No.: 13/624,390

(22) Filed: Sep. 21, 2012

(51) Int. Cl. B65D 81/24; A61M 35/00; B65B 5/04; B65B 55/00; B65D 81/26; B65D 75/00

(52) U.S. Cl. 206/204; 206/438; 206/205; 206/213.1; 53/428; 53/449; 53/431

(57) ABSTRACT

A transdermal drug delivery device is disclosed for administering an oxidizably and/or hydrolyzably degradable drug, e.g., cholinesterase inhibitors such as rivastigmine. The device comprises a) a substantially impermeable backing layer; b) an adhesive layer substantially free of antioxidant and containing a therapeutically effective amount of the degradable drug, which adhesive drug-containing layer is capable of adhering directly to a subject's skin or to another adhesive layer which is capable of adhering to a subject's skin; and c) a substantially impermeable protective release liner layer which releasably contacts the adhesive drug-containing layer or another adhesive layer. The delivery device is sealed in a degradation protective packaging system, such as a substantially oxygen impermeable heat sealable plastic pouch that contains a substantially inert gas and/or a degradation protectant material for the drug, such as an antioxidant, which can be inserted separately to the pouch. A method of making the device is also provided.

### FIELD

This invention relates to transdermal drug delivery devices and more particularly, to transdermal delivery devices for drugs which are subject to degradation during storage by hydrolysis and/or oxidation, e.g., rivastigmine.

### BACKGROUND

The transdermal route of parenteral drug delivery provides many advantages over other administrative routes. Transdermal drug delivery devices, including multilaminates and monoliths, for delivering a wide variety of drugs or other beneficial agents are described in U.S. Pat. Nos. 3,598,122; 3,598,123; 3,731,683; 3,797,494; 4,031,894; 4,201,211; 4,286,592; 4,314,557; 4,379,454; 4,435,180; 4,559,222; 4,568,343; 4,588,580; 4,645,502; 4,698,062; 4,704,282; 4,725,272; 4,781,924; 4,788,062; 4,816,258; 4,849,226; 4,904,475; 4,908,027; 4,917,895; 4,938,759; 4,943,435; 5,004,610; 5,071,656; 5,141,750; 5,342,623; 5,411,740; and 5,635,203.

One problem associated with the devices of the prior art is degradation of the pharmaceutical active ingredient, as well as certain contents of the device, such as the permeation enhancers, matrix materials, or other components. Degradation can result from both internal and external conditions. Internal conditions include the presence of inactive ingredients such as acid, base and oxidants, which may react with and degrade the active pharmaceutical ingredients (APIs). Impurities from these inactive ingredients not only undesirably break down these materials, but can also cause discoloration and formation of odors even within a system which separates the active ingredients from external conditions, such as heat, light, moisture and oxygen, e.g., by use of an envelope or pouch. Devices susceptible to degradation cannot be stored for a commercially reasonable amount of time, thus causing practical problems in their distribution.

Transdermal drug delivery systems typically comprise at a minimum a drug reservoir layer covered or surrounded by a backing layer and a release liner. The backing layer may be occlusive or non-occlusive. For example, the Climara® system comprises a polyethylene backing layer having a low moisture vapor transmission rate (MVTR) of approximately 7-11 g/m²·24 hr. More open backing layers such as spun laced polyester (Sontara®) are disclosed in U.S. Pat. Nos. 5,411,750, 5,500,222, and 5,614,211.

One solution to the degradation problem incorporates an antioxidant within the device. For example, U.S. Pat. Nos. 5,028,431 and 5,242,433, incorporated herein by reference in their entireties, disclose mixing antioxidants such as BHT into the drug formulation of a transdermal drug delivery device where the drug to be delivered exhibits instability. However, such systems suffer because the antioxidant reacts or mixes with the active ingredient, causing a decrease in purity of the active.

Even if placed in pouches containing degradation protectants such as antioxidants and desiccants, certain transdermal delivery devices still degrade at rates higher than desirable, particularly where their active pharmaceutical ingredient is unstable in the presence of moisture and/or oxygen. Thus, there is a need for improved storage stability of such devices.

A transdermal composition in the form of a patch is described in Example 2 of GB 2,203,040 according to which a degradable drug rivastigmine is mixed with two polymers and a plasticiser to form a viscous mass. This mass is applied to a foil which is cut into patches. Rivastigmine, its use and preparation are further disclosed in U.S. Pat. No. 4,948,807. The contents of UK patent application GB 2,203,040, and U.S. Pat. No. 4,948,807, are incorporated herein by reference in their entireties.

Rivastigmine is a reversible cholinesterase inhibitor (parasympathomimetic or cholinergic agent) that has been approved for the treatment of mild to moderate dementia of the Alzheimer's type, and mild to moderate dementia associated with Parkinson's disease. This drug has been shown effective for treatment of Alzheimer disease by oral route in a dose range of 6 to 12 mg per day. However, some patients are unable to tolerate oral administration, so alternate dosage techniques have been developed, including transdermal administration.

Pathological changes in dementia of the Alzheimer type and dementia associated with Parkinson's disease involve cholinergic neuronal pathways. While the precise mechanism of rivastigmine's action is unknown, it is postulated to exert its therapeutic effect by enhancing cholinergic function by increasing the concentration of acetylcholine through reversible inhibition of its hydrolysis by cholinesterase.

U.S. Pat. Nos. 6,316,023 and 6,335,031, both of which are hereby incorporated in their entirety by reference, disclose a transdermal device comprising an addition salt, 0.01 to 0.5 weight percent of an antioxidant and a diluent or carrier; a release liner; and an adhesive layer between the layer comprising (S)—N-ethyl-3-[(1-dimethylamino)ethyl]-N-methylphenyl carbamate (i.e., rivastigmine) in a polymer matrix and release liner. These references teach providing antioxidant within a polymer matrix containing rivastigmine active to prevent significant degradation over a long period of time. They also teach that forming an occlusive polymer matrix around the rivastigmine active ingredient and its storage in an air-tight package failed to reduce degradation in the absence of antioxidant.

Formulating an antioxidant with a drug molecule and polymer matrix in a single layer can be especially problematic if the antioxidant is chemically and/or physically incompatible with the drug molecule and/or the adhesive matrix. Accordingly, there is a need to provide a method to prevent degradation of active pharmaceutical ingredient present as a component of a transdermal delivery device, without resorting to including antioxidant within the matrix that contains the active. Phenylcarbamates such as rivastigmine have been found particularly susceptible to degradation during storage of the delivery device.

U.S. Pat. No. 6,660,295, incorporated in its entirety herein by reference, discloses a transdermal drug delivery device package comprising a backing layer; an adhesive matrix layer; a protective release liner layer; and an oxidative protective packaging system. The '295 patent teaches use of non-occluded backing layer to improve exposure of degradable components to a “degradation protectant” which exposure is otherwise limited to diffusing through the unsealed edges of the device. The reference teaches that “stability of such devices can be considerably improved when stored in pouches containing degradation protectants if the transdermal device uses a non-occlusive backing.” This method has limited utility for a transdermal delivery device because of undesired migration of the drug from an adhesive layer, and less than optimal skin hydration effects.

U.S. Published Application 2011/0151003 discloses a transdermal drug delivery device package which comprises a backing layer substantially impervious to the drug being delivered, a drug reservoir adhesive layer, a skin contact adhesive layer wherein each adhesive layer provides a different rate of drug delivery therefrom, and a release liner whose removal exposes the skin contact adhesive layer. The resulting dosage units are stored in appropriate packaging until used.

It would be desirable to provide a transdermal drug delivery device package which does not rely on “degradation protectant,” e.g., antioxidant, being exposed to degradable components through a non-occluded backing layer. In particular, it would be highly desirable to provide a transdermal drug delivery device package which does not add antioxidant to the layer containing active pharmaceutical ingredient, and whose external layers are impermeable to moisture, thereby preventing unwanted migration of the drug being administered, particularly if in liquid form, and improved hydration of the patient's skin at the site of the patch. It also would be advantageous to provide a transdermal drug delivery system which does not require active “degradation protectant” in the oxidative protective packaging system.

### SUMMARY

In one aspect, the present disclosure relates to a transdermal drug delivery device which comprises: a) a substantially impermeable backing layer; b) an adhesive drug-containing layer substantially free of antioxidant and containing a therapeutically effective amount of an oxidizably and/or hydrolyzably degradable drug, which adhesive drug-containing layer is capable of adhering directly to a patient's skin or indirectly via one or more optional intermediate layers at least one of which is another adhesive layer capable of adhering to a patient's skin; and c) a substantially impermeable protective release liner layer which releasably contacts the adhesive drug-containing layer or the another adhesive layer; and further wherein the delivery device is sealed within a substantially oxygen impermeable degradation protective packaging system.

In certain embodiments of this aspect of the drug delivery device of the disclosure, the packaging system comprises a degradation protectant. The degradation protectant can be selected from the group consisting of an inert gas, an antioxidant, an oxygen scavenger, a moisture scavenger, and a combination thereof.

In some embodiments of this aspect of the disclosure, the packaging system is filled with a substantially inert gas, e.g., nitrogen, and the oxygen level in the packaging system is no greater than about 5 wt. %.

In some embodiments of this aspect of the disclosure, the drug comprises an acetylcholinesterase inhibitor.

In certain embodiments of this aspect, the drug comprises a phenyl carbamate.

In various embodiments of this aspect, the drug is selected from the group consisting of rivastigmine in the form of a free base or rivastigmine in the form of an acid addition salt.

In some embodiments of this aspect, the drug is rivastigmine in the form of an acid addition salt, e.g. calcium tartrate.

In certain embodiments of this aspect, the drug is in a solid form under the conditions in which it is used. Such a drug can exhibit sufficient vapor pressure to provide effective transport, e.g., by sublimation, through the matrix to eventually reach the dermal surface.

In various embodiments of this aspect, the drug is in a liquid form under the conditions in which it is typically used. This would include drugs which are liquid by themselves under administration conditions, as well as solutions of such drugs in a liquid solvent, e.g., aqueous or non-aqueous solvents.

In some embodiments of this aspect, a) the substantially impermeable backing layer contains at least one of polyethylene terephthalate, nylon, polyethylene, polypropylene, polyester, polyester/ethylene-vinyl acetate, metallized polyester film, polyvinylidene chloride, metal foil, polyvinylidene fluoride film, ethylene vinyl acetate film laminated to a polyester, and ethylene vinyl acetate film laminated to a metallized polyester; b) the adhesive drug-containing layer contains i) at least one acrylic adhesive which is selected from acrylate co-polymer and cross-linked acrylate copolymer; ii) a cohesive promoter which is selected from polymers of methacrylate containing alkyl (C₁₋₄) ester groups, polymers of methacrylate esters containing trimethylaminoethyl cationic ester groups and other neutral (C₁₋₄) alkyl ester groups, a mixture of an acrylate polymer and a methacrylate polymer, polymers of acrylate esters containing methyl and ethyl neutral ester groups and trimethylaminoethyl cationic ester groups, iii) optionally, at least one intermediate layer comprising 1) at least one skin contact layer adhesive selected from silicone, silicone oil, natural rubber, synthetic rubber, polyisobutylene, neoprene, polybutadiene, polyisoprene, polysiloxane, cross-linked acrylic copolymer, uncross-linked acrylic copolymer, vinyl acetate adhesive, polyacrylate, ethylene vinyl acetate copolymer, styrene-isoprene copolymer, polyurethane, plasticized polyether block amide copolymer, plasticized styrene-rubber block copolymer, and mixtures thereof; 2) an optional drug component, and 3) an optional tackifier selected from pressure sensitive adhesives made from silicone polymer and resin, wherein the polymer to resin ratio provides a desired level of tack for adherence to a patient's skin; and iv) optionally, at least one intermediate layer comprising a membrane layer made of a flexible, polymeric material selected from low density polyethylene, high density polyethylene, ethylene vinyl acetate copolymers, and polypropylene; and c) the substantially impermeable protective release liner layer has a moisture vapor transmission rate (MVTR) of less than 20 g/m²·24 hr and comprises at least one of polyethylene terephthalate/silicone, polyethylene terephthalate/aluminized polyester coated with silicone, polyester with a silicone coating, polyurethane with a silicone coating, polyester with a fluorocarbon coating, polyurethane with a fluorocarbon coating, polyester with a fluorosilicone coating, polyurethane with a fluorosilicone coating, polyolefin coated with a fluoropolymer release agent, polyester coated with a fluoropolymer release agent, paper, thermoplastics, polyester film, and metal foil; and the substantially oxygen impermeable degradation protective packaging system has an oxygen transmission rate of less than about 0.05 ml/100 in²/24 hr/bar measured at 22° C. (72° F.), and comprising a sealable thermoplastic pouch containing an acrylonitrile-methyl acrylate copolymer, with the packaging system further comprising a degradation protectant selected from at least one of an inert gas, an antioxidant, an oxygen scavenger, and a moisture scavenger.

In yet other embodiments of this aspect, a) the substantially impermeable backing layer is a film containing at least one layer selected from polyethylene terephthalate, nylon, polyethylene, polypropylene, polyester, polyester/ethylene-vinyl acetate, and metallized polyester; b) the adhesive drug-containing layer contains i) at least one acrylic adhesive which is selected from a) uncross-linked copolymer comprising a first monomer selected from butyl acrylate, ethyl hexyl acrylate and vinyl acetate and a second monomer which differs from the first monomer; and b) cross-linked copolymer comprising a third monomer selected from butyl acrylate, ethyl hexyl acrylate and vinyl acetate and a fourth monomer different from the third monomer; ii) a cohesive promoter which is selected from polymers of methacrylate containing alkyl (C₁₋₄) ester groups, polymers of methacrylate esters containing trimethylaminoethyl cationic ester groups and other neutral (C₁₋₄) alkyl ester groups, and a mixture of an acrylate polymer and a methacrylate polymer; iii) optionally, at least one intermediate layer comprising 1) at least one skin contact layer adhesive selected from silicone, silicone oil, natural rubber, synthetic rubber, polyisobutylene, neoprene, polybutadiene, polyisoprene, polysiloxane, cross-linked acrylic copolymer, and uncross-linked acrylic copolymer; 2) an optional drug component, and 3) an optional tackifier selected from pressure sensitive adhesives made from silicone polymer and resin, wherein the polymer to resin ratio provides a desired level of tack for adherence to a patient's skin; iv) optionally, at least one intermediate layer comprising a membrane layer made of a flexible, polymeric material selected from low density polyethylene, high density polyethylene, ethylene vinyl acetate copolymers, and polypropylene; c) the substantially impermeable protective release liner layer having a moisture vapor transmission rate (MVTR) of less than about 15 g/m²·24 hr and comprises at least one of polyester film coated with a fluoropolymer release agent and polypropylene film coated with a fluoropolymer release agent; and the substantially oxygen impermeable degradation protective packaging system has an oxygen transmission rate of less than about 0.03 ml/100 in²/24 hr/bar measured at 22° C. (72° F.), and comprising a sealable pouch containing an oxygen impermeable, acrylonitrile-methyl acrylate copolymer film, the packaging system further comprising a degradation protectant selected from at least one of an inert gas, an antioxidant, an oxygen scavenger, and a moisture scavenger. The second monomer can include any monomer, including a monomer selected from butyl acrylate, ethyl hexyl acrylate and vinyl acetate, provided it is not the same as the first monomer. The fourth monomer can include any monomer, including a monomer selected from butyl acrylate, ethyl hexyl acrylate and vinyl acetate, provided it is not the same as the third monomer.

In still other embodiments of this aspect, a) the substantially impermeable backing layer comprises a three layer structure of polyethylene/polyurethane adhesive/polyethylene terephthalate; b) the adhesive drug-containing layer contains i) rivastigmine, ii) acrylate copolymer cohesive promoter, and iii) pressure sensitive adhesive comprising a copolymer of butyl acrylate, ethyl hexyl acrylate and vinyl acetate and the another adhesive layer capable of adhering to a patient's skin comprises a silicone oil tackifier and an amine compatible silicone adhesive; c) the substantially impermeable protective release liner layer contains a fluoropolymer coated polyester film; and the substantially oxygen impermeable degradation protective packaging system comprises a sealable multilayer pouch comprising, from its external surface, polyester film/adhesive/polyethylene film/aluminum foil/adhesive/heat-sealable, oxygen impermeable, acrylonitrile-methyl acrylate copolymer film, the packaging system further comprising a degradation protectant comprising nitrogen with an oxygen level in the pouch of no greater than about 5 wt. %.

In some embodiments of this aspect of the disclosure, the degradation protective packaging system comprises a sealable plastic layer which is sealable by at least one of heat, pressure, solvent, and adhesive.

In certain embodiments of this aspect, the degradation protective packaging system comprises a heat sealable plastic layer.

In various embodiments of this aspect, the adhesive drug-containing layer contains antioxidant at levels insufficient to stabilize the drug against degradation from oxidation and/or hydrolysis. Typically, the antioxidant is present in an amount of less than about 0.01 weight percent, e.g., less than about 0.005 weight percent.

In some embodiments of this aspect of the disclosure, each substantially impermeable layer comprises a moisture vapor transmission rate (MVTR) of less than 20 g/m²·24 hr, typically less than about 17 g/m²·24 hr, e.g., from about 1 g/m²·24 hr to about 15 g/m²·24 hr.

In another aspect, the disclosure relates to a method for preventing degradation of a transdermal drug delivery device of the type comprising a drug reservoir positioned between a backing layer and a release liner layer comprising: (a) providing the transdermal drug delivery device with a substantially impermeable backing layer and a substantially impermeable release liner each having a moisture vapor transmission rate of less than 20 g/m²·24 hr; (b) providing a degradation protectant within a substantially oxygen impermeable pouch or pouch precursor; (c) placing the device within the pouch or pouch precursor; and (d) sealing the pouch or pouch precursor. For present purposes, a pouch precursor can be a structure, which is converted to a pouch as a result of the sealing process, e.g., two flat sheets of pouching material, one atop the other, which are sealed around all sides to form a pouch. In certain embodiments, the patch can be sandwiched between two sheets having heat sealable surfaces facing each other. The pouch can be formed by heat-sealing surfaces around the patch.

In certain embodiments of this aspect, the drug reservoir comprises rivastigmine in the form of a free base or acid addition salt, e.g., rivastigmine tartrate.

In yet another aspect, the present disclosure relates to a method for preparing a transdermal drug delivery device of the type which is resistant to degradation of the drug during storage comprising: i) attaching a substantially impermeable backing layer to one side of an adhesive drug-containing layer substantially free of antioxidant and containing a therapeutically effective amount of an oxidizably and/or hydrolyzably degradable drug, which adhesive drug-containing layer is capable of adhering directly to a subject's skin or indirectly via one or more optional intermediate layers at least one of which is another adhesive layer capable of adhering to a subject's skin; ii) attaching to the other side of the adhesive drug-containing layer, or the optional another adhesive layer if present, a substantially impermeable protective release liner layer which releasably contacts the adhesive drug-containing layer or the another adhesive layer; and iii) sealing the product of steps i) and ii) within a substantially oxygen impermeable degradation protective packaging system.

In various embodiments of this aspect, the drug comprises rivastigmine in the form of a free base or acid addition salt.

In still another aspect, the present disclosure relates to a method for preparing a transdermal drug delivery device of the type which is resistant to degradation of the drug during storage. The method comprises i) coating a first release liner with a liquid precursor to a solid matrix reservoir layer containing an oxidizably and/or hydrolyzably degradable drug and substantially free of antioxidant; ii) drying the liquid precursor to provide a solid matrix reservoir layer-coated first release liner; iii) laminating the coated side of the solid matrix reservoir layer-coated first release liner to a substantially impermeable backing layer; iv) removing the first release liner to provide an exposed solid matrix reservoir layer surface; v) coating a second release liner with a liquid precursor to a solid adhesive skin contact layer; vi) drying the liquid precursor of step (v) to provide a solid adhesive skin contact layer-coated release liner having an exposed solid adhesive skin contact layer surface; vii) laminating the exposed solid adhesive skin contact layer surface of step vi) to the exposed solid matrix reservoir layer surface of step iv) to provide a multi-laminate comprising a) an external backing layer, b) a solid matrix reservoir, c) a solid adhesive skin contact layer, and d) a release liner layer; viii) slitting and/or die cutting the multi-laminate to provide an individual patch of desired width and/or shape; and ix) individually sealing the patch within a substantially oxygen impermeable degradation protective packaging system. Components (a) through (d) are in relative configuration to each other, with a) external backing layer facing outside the patient when worn, while release liner layer d) is on the opposite side, which when removed allows direct contact of the skin with c) skin contact layer.

### BRIEF DESCRIPTION OF THE DRAWINGS

FIG. 1 depicts a schematic diagram of the product of the present disclosure which has a single intermediate layer positioned between a backing film upper layer and lower layer comprising a protective release liner. The intermediate layer comprises a monolithic adhesive matrix which comprises a) acrylic adhesive, b) active pharmaceutical ingredient, e.g., rivastigmine and c) cohesive promoter.

FIG. 2 depicts a schematic diagram of the product of the present disclosure which has multiple intermediate layers positioned between a backing film upper layer and lower layer comprising protective release liner. The intermediate layer comprises a multiple drug reservoir layer, i.e., a bilayer comprising A) an upper acrylic adhesive matrix layer comprising i) acrylic adhesive, ii) active pharmaceutical ingredient (rivastigmine), and iii) adhesive promoter and B) a lower skin contact layer comprising i) silicone adhesive, ii) optional active pharmaceutical ingredient, e.g., rivastigmine, and iii) tackifier.

FIG. 3 depicts an overhead view of an embodiment of the transdermal drug delivery device of the present disclosure.

FIG. 4 depicts a side view of an embodiment of the transdermal drug delivery device of the present disclosure.

### DETAILED DESCRIPTION

This disclosure relates to a pharmaceutical composition for transdermal administration of an oxidizably and/or hydrolyzably degradable drug. The drug can be a phenyl carbamate, which is useful in inhibiting acetylcholinesterase in the central nervous system, e.g. for the treatment of Alzheimer's disease. In one embodiment, the phenyl carbamate can be rivastigmine, also known as (S)—N-ethyl-3-[1-dimethylamino)ethyl]-N-methyl-phenyl-carbamate, which can be provided in free base or acid addition salt form.

Phenyl carbamates, e.g., rivastigmine, have been shown as susceptible to degradation, particularly in the presence of oxygen. The transdermal composition described in GB 2,203,040 has been found to degrade, possibly by oxidative degradation, despite the formation of an occlusive polymer matrix around rivastigmine and its storage in air-tight packaging.

The subject matter disclosed herein relates to transdermal drug delivery device used for introducing a drug which is degradable by exposure to ambient conditions such as air and moisture. The subject matter further relates generally to a method for transdermal drug delivery for rivastigmine and related cholinesterase inhibitors, which are typically subject to degradation by oxidation, e.g., by auto oxidation which occurs spontaneously at room temperature, and/or hydrolysis. In addition to rivastigmine, suitable acetylcholinesterase inhibitors for use in the present invention include donepezil and galantamine.

Oxidation can be a prime cause of product instability, often by adding oxygen or removing hydrogen. Redox potential can provide valuable predictive information for a particular drug. Examples of oxidation susceptible drugs include epinephrine, phenylephrine, lincomycin, isoprenaline, and procaine hydrochloride. Forced degradation testing of a drug can be carried out in 3% hydrogen peroxide solution for 24 hours. After this time if 50 wt. % or greater of the drug is found to degrade under these free-radical oxidative conditions, the drug will be considered susceptible to degradation by oxidation.

Hydrolysis will often occur with drugs that contain an ester or amide linkage. Examples of such drugs include cocaine, physostigmine, procaine, tetracaine, thiamine, benzocaine, and benzylpenicillin. Forced degradation testing of a drug can be carried out in 0.5 N NaOH for 24 hours. If 15 wt. % or greater of the drug is found to degrade under these base hydrolysis conditions, the drug will be considered susceptible to degradation by hydrolysis.

It has now been found that stable pharmaceutical compositions of the present disclosure comprising an oxidizably and/or hydrolyzably degradable drug compound, such as phenyl carbamate, e.g., rivastigmine, show less than significant degradation of the drug compound over a prolonged time period.

The transdermal drug delivery devices of the present disclosure show a reduction in degradation by-products in stress stability tests. The devices of the invention may contain high amounts of active pharmaceutical ingredient such as rivastigmine, typically from about 1 to about 40% by weight, e.g., about 10 to 35%, more particularly from about 20 to about 35%, e.g. about 30%.

The substantially impermeable backing layer is a flexible substrate, e.g., a film or laminate, which provides support for the rest of the transdermal drug delivery device during storage, handling and wear. Any backing layer of sufficient strength and rigidity, and which is substantially impermeable to the active pharmaceutical ingredient or ingredients present in the transdermal drug delivery device of the present disclosure, is suited for use in the present disclosure. For present purposes, “substantially impermeable” means that no substantial loss of active ingredient from the backing layer occurs under typical storage and usage conditions and periods which the device is expected to encounter during its lifetime. Typically, such losses are less than about 1 wt. %, preferably less than about 0.1 wt %, or even less than about 0.01 wt. %. The backing layer can be substantially moisture impermeable as well. Typically, a backing layer of the present disclosure has a moisture vapor transmission rate (MVTR) of less than 20 g/m²·24 hr, typically less than about 17 g/m²·24 hr, e.g., less than about 15 g/m²·24 hr, e.g., from about 1 g/m²·24 hr to about 15 g/m²·24 hr.

For present purposes, MVTR is defined as a measure of the passage of water vapor through a substance. It can be measured using suitable techniques. ASTM F1249 describes the procedure for MVTR testing using modulated infrared sensors which require use of a known calibration standard and determining the voltage-to-transmission-rate ratio for each sensor. Coulometric sensors for measuring oxygen transmission rates in flat films and packages, following ASTM D3985 and F1307 are also suitable and require no calibration. Typical rates in aluminum foil laminates may be as low as 0.001 g/m²/day, whereas the rate in fabrics can measure up to several thousand g/m²/day.

In various embodiments of the present disclosure, the backing layer is composed of materials that are selected from polyesters, e.g., polyethylene terephthalate, various nylons, polypropylenes, polyester/ethylene-vinyl acetates, metallized polyester films, polyvinylidene chloride, metal films such as aluminum foils, polyvinylidene fluoride films, or mixtures or copolymers thereof.

Other non-limiting materials for the backing layer include ethylene vinyl acetate films laminated to a polyester, ethylene vinyl acetate films laminated to a metallized polyester, MEDIFLEX® 1200, MEDIFLEX® 1501, MEDIFLEX® 1505, MEDIFLEX® 1201, MEDIFLEX® 1502 (all five MEDIFLEX® products being available from Mylan Technologies Inc., St. Albans, Vt., USA), DuPont polyester type S available from DuPont, Wilmington, Del., USA, Dow BLF® 2050 available from The Dow Chemical Company, Midland, Mich., USA, and 3M™ Scotchpak™ 1109, 3M™ Scotchpak™ 9723, 3M™ Scotchpak™ 9733, 3M™ Scotchpak™ 9735 and 3M™ Scotchpak™ 9730, which Scotchpak™ products are available from 3M of Minneapolis, Minn., USA.

Additional materials suited for the backing layer include polyethylene or polyolefin backings, such as MEDIFLEX® 1000, 3M™ CoTran™ 9722, and 3M™ CoTran™ 9720. 3M™ CoTran™ products are available from 3M of Minneapolis, Minn., USA.

In some embodiments of the disclosure, the backing layer comprises a film selected from MEDIFLEX® 1501 (which has an MVTR of 14 g/m²·24 hr), MEDIFLEX® 1000 (which has an MVTR of 7 g/m²·24 hr), available from Mylan Technologies Inc., Scotchpak™ 1109, Scotchpak™ 9730, Scotchpak™ 9732, Scotchpak™ 9733, Scotchpak™ 9735, as well as CoTran™ 9719, CoTran 9720, and CoTran 9726, available from 3M. The Scotchpak™ and CoTran films exhibit a relatively low MVTR range of from about 0.5 to about 17 g/m²·24 hr.

In certain embodiments, the backing layer is comprised of ethylene vinyl acetate films laminated to a polyester, such as MEDIFLEX® 1501 from Mylan Technologies, Inc. MEDIFLEX® 1501 is a three layer structure which can be described from its external surface inwardly as follows: peach colored polyethylene/polyurethane adhesive/polyester.

In some embodiments, the backing layer may be the same size as the adhesive drug-containing layer. In other embodiments, the backing layer may be oversized as compared with the adhesive drug-containing layer, i.e., the backing layer may be larger than the adhesive drug-containing layer.

In certain embodiments of the present disclosure, the backing layer may range from about 0.01 mm to at least 10 mm larger than the adhesive drug-containing layer, preferably ranging from about 0.05 mm to about 5 mm larger than the adhesive drug-containing layer, and most preferably ranging from about 0.1 mm to about 3 mm larger than the adhesive drug-containing layer. Without wishing to be bound by any particular theory, it is believed that the use of an oversized backing layer helps prevent the adhesive drug-containing layer and the remaining portions of the transdermal drug delivery device of the present disclosure from becoming distorted or relaxing during the handling and/or shipping processes.

The backing layer should be thick enough to resist wrinkling which may arise upon prolonged periods in storage and through the movement of a subject's skin. Typically, the backing layer is, from about 50 microns to about 100 microns in thickness.

An adhesive drug-containing layer (or drug reservoir layer) substantially free of antioxidant and containing a therapeutically effective amount of an oxidizably and/or hydrolyzably degradable drug, is placed directly or indirectly (through an intermediate layer) on the front side of the backing layer, i.e., the side toward the wearer, in the transdermal drug delivery device of the present disclosure.

In various embodiments of the present disclosure, the adhesive drug-containing layer comprises an acrylic adhesive and active pharmaceutical ingredient, e.g., rivastigmine in its free base or acid addition salt form. In addition, the adhesive drug-containing layer can contain an optional cohesive promoter. Typically, this adhesive drug-containing layer comprises acrylic adhesive in amounts ranging from about 40 wt. % to about 99 wt. %, active pharmaceutical ingredient in amounts ranging from about 1 wt. % to about 40 wt. %, and cohesive promoter in amounts ranging from about 0 wt. % to about 30 wt. %.

In certain embodiments of the present disclosure, this layer comprises acrylic adhesive in amounts ranging from about 50 wt. % to about 75 wt. %, active pharmaceutical ingredient in amounts ranging from about 15 wt. % to about 25 wt. %, and cohesive promoter in amounts ranging from about 15 wt. % to about 25 wt. %.

In other embodiments of the present disclosure, the adhesive drug containing layer comprises acrylic adhesive in amounts ranging from about 45 wt. % to about 55 wt. %, active pharmaceutical ingredient in amounts ranging from about 17 wt. % to about 23 wt. %, and cohesive promoter in amounts ranging from about 17 wt. % to about 23 wt. %.

In certain embodiments, the acrylic adhesive can be selected from the group consisting of Duro-Tak™ 87-2352, Duro-Tak™-387-2353, Duro-Tak™ 87-235A, Duro-Tak™ 387-235A, Duro-Tak™ 87-2516, Duro-Tak™-387-2526, Duro-Tak™-87-2287, Duro-Tak™ 387-2287, Duro-Tak™ 87-2194, Duro-Tak™ 387-2051, Duro-Tak™ 387-2052, Duro-Tak™ 387-2194, Duro-Tak™ 87-2196, GMS-9073, GMS-2873, GMS-9083, GMS-2883, GS-9067, GMS-9071, GMS-3083, GMS-3253, GMS-737, GMS-737-01, GMS-788, GMS-2999, GMS-2495, GMS-7883, GMS-1753, GMS-2893 and combination thereof. The Duro-Tak™ products are available from Henkel of Dusseldorf, Germany and can be characterized as containing acrylic copolymers. GMS products are available from the Cytec Corporation Germany.

In various embodiments of the present disclosure, the acrylic adhesive comprises acrylate co-polymer, e.g. co-polymer of butyl acrylate, ethyl hexyl acrylate and vinyl acetate. The co-polymer can be cross-linked. A preferred acrylate polymer can be selected from the Duro-Tak™ brand, e.g. Duro-Tak™ 87-2353. Duro-Tak™ 87-235A is particularly advantageous inasmuch as it lacks the monomer glycidyl methacrylate component found in Duro-Tak™ 87-2353.

The acrylic adhesive layer can be attached or adjacent to a backing film directly or through a suitable intermediate layer. In certain embodiments of the present disclosure, the acrylic adhesives used in the intermediate layer are selected from Duro-Tak™ 87-2352, Duro-Tak™ 387-2353, Duro-Tak™ 87-235A, Duro-Tak™ 387-2351, Duro-Tak™ 87-2516, Duro-Tak™ 387-2526, Duro-Tak™ 87-2287, Duro-Tak™ 387-2287, Duro-Tak™ 87-2194, Duro-Tak™ 387-2194, Duro-Tak™ 87-2196 and combinations thereof.

For present purposes, the term “cohesive promoter” is defined as an additive to a layer which improves flexibility at low temperatures and compatibility of the various components of a particular layer.

In certain embodiments of the present disclosure, the cohesive promoter is selected from polymers of methacrylate containing alkyl (C₁₋₄) ester groups. In some embodiments, the polymer matrix is a mixture of an acrylate polymer and a methacrylate polymer e.g. in a weight ratio of from about 5:1 to about 1:1, e.g. about 4:1 to about 2:1 e.g. about 3:1, e.g. butylmethylacrylate and methylmethylacrylate. The cohesive promoter typically has a mean molecular weight of about 150,000. In one embodiment, the cohesive promoter comprises the acrylate copolymer Plastoid® B available from Evonik DeGussa GmbH of Germany.

In certain embodiments of the disclosure the cohesive promoter comprises polymers of acrylate and methacrylate esters containing methyl and ethyl neutral ester groups and trimethylaminoethyl cationic ester groups. Chloride ions may be present. In some embodiments the cohesive promoter can have a mean molecular weight of about 150,000, a maximum viscosity at 20° C. of about 15 cP, a refractive index of 1.380-1.385, a density of 0.815-0.835 g/cm³, and a ratio of cationic ester groups to neutral alkyl groups of 1:20 giving an alkali count of 28.1 mg KOH per gram polymer (Eudragit RL 100® available from Rohm) or 1:40 giving an alkali count of 15.2 mg KOH per gram polymer (Eudragit RS 100®, also available from Rohm).

In some embodiments of the disclosure the cohesive promoter comprises polymers of methacrylate esters containing trimethylaminoethyl cationic ester groups and other neutral (C₁₋₄) alkyl ester groups. Chloride ions can be present. In specific embodiments, the cohesive promoter has a mean molecular weight of about 150,000, a viscosity at 20° C. of about 10 cP, a refractive index of 1.38, a density of 0.815 g/cm³, a ratio of cationic ester groups to neutral alkyl groups of 1:20 giving an alkali count of 180 mg KOH per gram polymer. In some embodiments, the cohesive promoter comprises Eudragit E 100®, also available from Rohm.

In certain embodiments of the present disclosure, cohesive promoters are selected from Plastoid® B, Eudragit E and ethyl cellulose.

The devices of the present disclosure are suitable for the transdermal delivery of a wide range of drugs. Such drugs can be present within the adhesive layer or skin contact layer in solid form or liquid form. The present disclosure is particularly directed to drugs which are subject to degradation by hydrolysis and/or oxidation.

The term “drugs” is intended to have its broadest interpretation as including any therapeutically, prophylactically and/or pharmacologically or physiologically beneficial active substance, or a mixture thereof, which is delivered to a living being to produce a desired, beneficial effect. More specifically, any drug which can produce a pharmacological response, localized or systemic, whether therapeutic, diagnostic, or prophylactic in nature, is within the contemplation of the present invention. Also included within the scope of the invention are bioactive agents, such as insect repellants, sun screens, cosmetic agents, etc. The drug can be provided in an amount sufficient to cure, diagnose, or treat a disease or other condition. This definition includes, but is not limited to: 1. cardiovascular drugs, such as nitroglycerin, propranolol, isosorbide dinitrate, isosorbide mononitrates, diltiazem, nifedipine, procainamide, clonidine and others, 2. androgenic steroids, such as testosterone, methyltestosterone and fluoxymesterone, 3. estrogens, such as conjugated estrogens, esterified estrogens, etropipate, 17-β estradiol, 17-β estradiol valerate, equilin, mestranol, estrone, estriol and diethylstilbestrol, 4. progestational agents, such as progesterone, 19-norprogesterone, norethindrone, norethindrone acetate, melengestrol chloradinone, ethisterone, medroxyprogesterone acetate, hydroxyprogesterone caproate, norethynodrel, dimethisterone, ethinylestrenol, norgestrel, megestrolacetate, and ethinodiol diacetate, 5. drugs which act on the central nervous system, including sedatives, hypnotics, analgesics, anesthetics, and antianxiety agents; such as salicylic acid derivatives, opiates, opioids and the like; including chloral hydrate, benzodiazepines, naloxone, haloperidol, pentobarbitol, phenobarbitol, secobarbital, codeine, lidocaine, dibucaine, benzocaine, fentanyl, fentanyl analogs and nicotine, 6. nutritional agents, including vitamins, essential amino acids and essential fats, 7. anti-inflammatory agents, including hydrocortisone, cortisone, dexamethasone, prednisolone, prednisone, halcinonide, methylprednisolone, fluorocortisone, corticosterone, paramethasone, ibuprofen, naproxen, fenoprofen, fenbufen, indoprofen, salicylic acid, methyl salicylate, sulindac, mefenamic acid, piroxicam, indonisilone and tolmetin, 8. antihistamines, such as diphenhydramine, triprolidine, chlorcyclizine, promethazine, cyclizine, chlorprenaline, terrenadine, phenylpropanolamine and chlorpheniramine, 9. miotics, such as pilocarpine, 10. dermatological agents, such as vitamins A and E, 11. anti-spasmodics, including atropine, methantheline, papverine, cinnmedrine and methscopolamine, 12. anti-depressants, such as isocaboxazid, phenelzine, imipramine, amitrptyline, trimepramine, dozepin, desipramine, nortriptyline, protriptyline, amoxapine and maprotiline, 13. anti-cancer drugs, 14. anti-diabetics, such as insulin, 15. anti-estrogens or hormone agents, including tamoxifen or HCG, 16. anti-infectives, including antibiotics, anti-bacterials and anti-virals, such as tetracycline, chloramphenicol, sulfacetamide, sulfadiazine, sulfamerazine, sulfoxazole, idoxuridine, and erythromycin, 17. anti-allergenics, such as antazoline, metapyrilene, and pyrilamine, 18. anti-pyretics, including aspirin and salicylamide, 19. anti-migraine agents, including dihydroergotamine and pizotyline, 20. tranquilizers, including reserpine, chlorpromazine, and antianxiety benzodiazepines, and 21. anti-psychotic agents, including haloperidol loxapine, molindone, thiothixene, pimozide, risperidone, quetiapine fumarate, olanzapine, and/phenothiazine derivatives.

Other drugs suitable for delivery using a transdermal system can be readily determined by persons of ordinary skill in the art. In addition, pharmacologically acceptable derivatives of the drugs, such as ethers, esters, amides, acetals, salts and the like, which are suitable for transdermal administration can be used.

In certain embodiments of the disclosure, a device or composition of this invention comprises a drug which acts as a cholinesterase inhibitor, e.g., an acetylcholinesterase inhibitor, such as one selected from rivastigmine, donepezil and galantamine.

In particular embodiments of the present disclosure, the drug can be selected from the group consisting of (S)—N-ethyl-3-[(1-dimethylamino)ethyl]-N-methylphenyl carbamate in the form of a free base or acid addition salt.

In one specific embodiment, the drug comprises (S)—N-ethyl-3-[(1-dimethylamino)ethyl]-N-methylphenyl carbamate in the form of a tartrate salt, e.g., calcium tartrate salt.

In certain embodiments of the present disclosure, the transdermal drug delivery device comprises plural or multiple adhesive drug-containing layers, e.g., adhesive bilayers, trilayers, quadrilayer, pentalayers, etc., which include an acrylic adhesive matrix layer and a skin contact layer. The skin contact layer can be directly attached to the acrylic adhesive matrix layer, or alternately, is attached to an intermediate membrane layer placed between the acrylic adhesive matrix layer and the skin contact layer. In plural or multiple adhesive drug-containing layer embodiments of the present disclosure, a skin contact layer is typically present in addition to a separate acrylic adhesive layer as described above. In some embodiments the skin contact layer itself contains an active pharmaceutical ingredient. In other embodiments, no active pharmaceutical ingredient is present in the skin contact layer.

The skin contact layer adjacent to the adhesive drug containing layer comprises a skin contact layer adhesive, an optional drug component, usually of the type already present in the adhesive layer, and an optional tackifier.

This adhesive is selected from silicones, including silicone oils, e.g., “medical fluids,” natural and synthetic rubbers, polyisobutylene (“PIB”) (HMW (High Molecular Weight) PIB, LMW (Low Molecular Weight) PIB, or mixtures of HMW and LMW PIB), neoprenes, polybutadienes, polyisoprenes, polysiloxanes, acrylic adhesives including cross-linked and uncross-linked acrylic copolymers, vinyl acetate adhesives, polyacrylates, ethylene vinyl acetate copolymers, styrene-isoprene copolymers, polyurethanes, plasticized polyether block amide copolymers, plasticized styrene-rubber block copolymers, and mixtures thereof.

In some embodiments, the skin-contact layer contains a mixture of high molecular weight polyisobutylene (HMW PIB) and low molecular weight polyisobutylene (LMW PIB).

In certain of such embodiments, the skin contact layer comprises a silicone adhesive, active pharmaceutical ingredient, and an optional tackifier.

In some embodiments, the skin contact layer comprises suitable silicone adhesives include pressure sensitive adhesives made from silicone polymer and resin. The polymer to resin ratio can be varied to achieve different levels of tack. Specific examples of useful silicone adhesives for this purpose which are commercially available include the standard Dow Corning® BIO-PSA series (7-4400, 7-4500 and 7-4600 series) and the amine compatible (endcapped) Dow Corning® BIO-PSA series (7-4100, 7-4200 and 7-4300 series) manufactured by Dow Corning. Preferred adhesives include Dow Corning® BIO-PSA 7-4202, 7-4301, 7-4302, 7-4501, 7-4502 and 7-4602.

In certain embodiments, the skin-contact layer contains an active pharmaceutical ingredient (API), where the API itself and degree of saturation of API in this layer is about the same as the degree of saturation in the adhesive layer.

In some embodiments, the skin-contact layer contains one or more additives, e.g., a tackifier. A tackifier is added to provide a desired stickiness to the product, typically a stickiness sufficient to adhere the device to a patient's skin, without the patient feeling undue discomfort upon removal of the device. Tack can be measured by conventional techniques such as the Instron Peel Strength Test or the “Tel-Tak” test, employing a tackmeter sold by Monsanto Co., Testing Instruments Division.

In various embodiments of the present disclosure, the optional tackifier can also be present in the adhesive drug-containing layer, typically in amounts ranging from about 10 wt. % to about 30 wt. %, e.g., from about 15 wt. % to about 25 wt. %, e.g., from about 17 wt. % to about 23 wt. %, typically, about 20 wt. %. The tackifier can be a suitable silicone oil, e.g., polydimethyl siloxane, such as Dow Corning® Medical Fluid, 12,500 cSt.

Intermediate Membrane

Transdermal delivery devices manufactured according to the present disclosure may further comprise a membrane layer. Typically, a membrane layer, if included, is located between a skin contact layer and the drug reservoir layer (the skin-contact layer being adjacent to the release liner and the drug reservoir layer being adjacent to the backing layer).

The membrane layer may serve a variety of purposes, such as slowing diffusion of the API(s) or providing structural integrity for the patch. The membrane layer is selected such that it is permeable with respect to the API(s) yet may change the skin penetration profile of the device compared to a like device not having the membrane.

Suitable membranes include solid, nonporous film membranes and membranes with physical pores or channels. The membrane is preferably made of flexible, polymeric materials used conventionally by those skilled in the art. Polymer films which may be used for making the membrane layer include, without limitation, those comprising low density polyethylene, high density polyethylene, ethylene vinyl acetate copolymers, polypropylene and other suitable polymers.

The membrane thickness can generally range from about 6 microns to about 100 microns (about 0.25 mil to about 4 mil). In some embodiments of the disclosure, the thickness can range from about 18 microns to about 33 microns (about 0.7 mil to about 1.3 mil).

One skilled in the art would be able to select an appropriate membrane layer by varying its size, shape, thickness, position relative to the skin, material, porosity, etc. to provide optimal delivery rates of the API to the skin.

Substantially impermeable protective release liners which are well known in the art can be used in the present invention provided they exhibit adequate moisture impermeability. For present purposes, “substantially impermeable” means that no substantial loss of active ingredient from the release liner occurs under typical storage and usage conditions and periods which the device is expected to encounter during its lifetime. Typically, such losses are less than about 1 wt. %, preferably less than about 0.1 wt %, or even less than about 0.01 wt. %. The release liner layer can be substantially moisture impermeable as well. For present purposes, the substantially impermeable release liner layer is a flexible substrate, e.g., a film or laminate, which is releasably secured to the skin contact layer, e.g., a peelable layer. The release layer serves to protect the adhesive surface of the layer of the device which is to be applied to a subject's skin, e.g., during storage and any other time prior to use of the device. Any release layer of sufficient strength and rigidity, and which is substantially moisture impermeable, is suited for use in the present disclosure. Typically, such a release liner layer has a moisture vapor transmission rate (MVTR) of less than 20 g/m²·24 hr, e.g., less than about 17 g/m²·24 hr, e.g., from about 1 g/m²·24 hr g/m²/24 hr to about 15 g/m²·24 hr. MVTR can be measured for the release liner in the same way as earlier described in relation to the backing layer.

Non-limiting examples of materials from which the release liner may be composed include polyethylene terephthalate/silicone (i.e. polydimethyl siloxane or “PET/SI”), polyethylene terephthalate/aluminized polyester coated with silicone (i.e. polydimethyl siloxane or “PET/MET/SI”), polyester or polyurethane liners with a silicone coating, polyester or polyurethane liners with a fluorocarbon or fluorosilicone coating, e.g., a polyfluoroalkylsiloxane, or polyester or polyurethane liners with a silicone coating.

In various embodiments, the protective release liner layer comprises fluorosilicone coated polyester or silicone coated polyester. Suitable release liners include polyester film and polypropylene film coated with a fluoropolymer release agent. Such release liner layers include Scotchpak™ 1020, 1022, 9741, 9742, 9744 and 9755 available from 3M, as well as MEDIRELEASE® 2500, MEDIRELEASE® 2249 and MEDIRELEASE® 2226, each of which is available from Mylan Technologies, Inc. Other release liner layers include, CPFilms Inc. Clearsil® UV5A and CPFilms Inc., Clearsil® UV510, CPFilms Inc. Sil® UV5A and CPFilms Inc. Sil® UV510, which are available from CPFilms, Inc. of Fieldale, Va., USA.

The release liner can, however, comprise other materials, including paper or paper-containing layers or laminates, various thermoplastics, polyester films, foil liners, and the like. The release liner is removed and discarded from the transdermal delivery device to expose the skin contact adhesive layer which functions as the means of adhering the composition to the patient and through which the drug passes as it is delivered to the patient. Suitable release liners include those known in the art for use with pressure sensitive adhesive compositions.

In some embodiments, the release liner may be the same size as the adhesive matrix layer and/or may be the same size as the backing layer. In any event, the release liner should be at least coextensive with the surface of the adhesive matrix layer, in order to prevent migration of the active component prior to release liner removal. In other embodiments, the release liner may be larger than the adhesive matrix layer and/or may be larger than the backing layer. In yet other embodiments, the release liner may range from about 0.1 mm to at least about 20 mm larger than the margin of a backing layer or an adhesive matrix layer, preferably ranging from about 0.5 mm to about 10 mm larger than the backing layer or adhesive matrix layer, and most preferably ranging from about 1 mm to about 5 mm larger than the backing layer or adhesive matrix layer. It is believed that the use of an oversized release liner facilitates its removal by the user prior to application to the skin and also helps prevent the adhesive matrix from becoming distorted or relaxing during the handling and shipping processes. In certain embodiments, the release liner is of square or rectangular shape and can be attached to a smaller patch, e.g., one of circular shape.

The dosage unit forms are made from the resulting multiple layer structure by die stamping to provide patches of desired shape and size.

Once the dosage unit forms have been prepared, they are placed in an appropriate packaging system for storage and sealed therein in the substantial absence of oxidizing agents, e.g., air, until they are to be used in transdermal treatment. The packaging system can comprise a pouch, envelope, or any other suitable structure surrounding the dosage unit form. The structure is typically made from a plastic film or laminated film which is substantially impermeable to water vapor and air, including oxygen. In various embodiments the laminate can comprise a thermoplastic film which is heat sealable.

In certain embodiments the packaging system comprises a pouch made from a multiple laminate film. The film typically exhibits an oxygen transmission rate of less than about 0.05 ml/100 in²/24 hr/bar measured at 22° C. (72° F.), e.g., less than about 0.04 ml/100 in²/24 hr/bar measured at 22° C. (72° F.), e.g., from about 0.01 to about 0.03 ml/100 in²/24 hr/bar.

Once the dosage unit forms have been prepared, they are placed and sealed in appropriate packaging for storage until they are to be applied in transdermal treatment.

The compositions of this disclosure possess sufficient adhesive properties that once the release liner is removed and the composition is applied to a patient's skin the composition can remain in place for a period of time sufficient to distribute the desired amount of the drug contained therein with a low incidence of debonding or delamination.

In another embodiment, the degradation protective packaging system in the present disclosure comprises an oxygen absorbent label attached to the inner side of a heat sealed pouch or an antioxidant packet inside a heat sealed pouch. A suitable example of the oxygen absorbent label is StabilOx® D20-H31. Examples of oxygen absorbent packets or sachets are StabilOx® D100-H31, StabilOx® D100-H42, StabilOx® F100-H60, PharmaKeep®-CH, and PharmaKeep®-KH. StabilOx® products are available from Multisorb Technologies, Buffalo, N.Y., USA, while PharmaKeep® products are available from Sud-Chemie, Munich, Germany, a division of Mitsubishi Gas Chemical Co., Inc.

In certain embodiments of the present disclosure, oxygen scavengers associated with the packaging system may themselves be further modified. An oxygen absorbent label or oxygen absorbent packet may be enclosed with a semipermeable film. Such a semipermeable film enclosure is substantially impermeable to the drug molecule or active pharmaceutical ingredient found in the transdermal patch, but substantially permeable to oxygen. The semipermeable film enclosure thus reduces or eliminates the undesirable adsorption or absorption of the drug molecules by the antioxidant label or packet, while at the same time reducing or eliminating oxidation of the drug molecule by contact with oxygen.

In an embodiment of the disclosure, an especially suitable semipermeable film for such use is heat sealable Barex® 210 film, available from INEOS Barex, a division of INEOS USA LLC of Newark, Del., USA. Barex® 210 film is an impact modified acrylonitrile-methyl acrylate copolymer with excellent gas barrier properties and a wide range of chemical resistances.

In various embodiments of the present disclosure, the oxygen scavenger material in the packaging system can be supplemented or replaced with a substantially inert gas. Such gases can include nitrogen, neon, and argon, and krypton, with nitrogen being preferred. The gas is used to flush oxidants, e.g., oxygen containing gas mixtures, from the packaging system prior to sealing in the drug delivery device. Such a system can rely solely on the addition of an inert gas to the packaging system, e.g., a protective envelope or pouch, in order to substantially displace the oxygen-containing gas with inert gas. After flushing, the packaging system (envelope or pouch) should contain no greater than about 5 wt. % oxygen or other oxidant, e.g., no greater than about 4 wt. %.

The drug delivery devices of this disclosure can be made by first preparing separate adhesive blends for each layer of the dosage unit, then dissolving or suspending the drug of choice in at least one of the blends, each of which has been made by mixing a suitable solvent with the pressure sensitive adhesive of choice. The drug reservoir layer can be coated first on a release liner, dried and then laminated to the desired backing film, according to predetermined parameters, such as temperature and dwell time (line speed), which yield minimal residual solvent levels. The skin contact layer can then be coated on a separate release liner and dried. The release liner can be removed from the drug reservoir layer and the adhesive side of the skin contact layer laminated onto the adhesive side of the drug reservoir layer so that the drug reservoir layer lies between the backing and the skin contact layer. If the drug initially is suspended or dissolved in only one of the two adhesive layers, it will, over time, equilibrate into the other adhesive layer until the degree of saturation is the same in both layers. It may be desirable to prepare the composition with the drug initially suspended or dispersed in only one of the two adhesive layers if, for example, the other adhesive layer is prepared with a solvent which would be deleterious to the drug but which evaporates during processing (coating and drying).

If more than two layers are to be provided, the third (middle) layer can be coated as a liquid onto a release liner, dried, laminated to either the adhesive side of the dried skin contact layer or the adhesive side of the dried drug reservoir layer once the release liner has been removed from the latter. Then the two parts of the dosage unit can be laminated to one another as above.

Suitable solvents for use in preparing the adhesive blends include acetone, heptane, ethyl acetate, isopropanol, ethanol, hexane, toluene, xylene, 2,4-pentanedione, methanol and water.

Alternative methods for producing or achieving a transdermal delivery dosage unit in accordance with this invention may be apparent to persons skilled in the art, and such alternative methods also fall within the scope of the present invention. For example, an adhesive blend can be coated onto the backing film rather than the release liner. Alternatively, an adhesive coating can be created without using a solvent, such as by heating a hot-melt adhesive to its melting temperature. With this technique, no drying of the adhesive is required, only cooling.

There are many coating techniques for applying a continuous liquid coating onto a substrate, including using a gravure roll, reverse roll, falling film, inkjet, etc. All of these are well-known to persons of ordinary skill in the art and can be used to create pressure-sensitive adhesive layers from a fluid blend. Alternatively, a thin adhesive coating can be achieved by extrusion, in which the adhesive blend is forced through a die under pressure onto the substrate either as a continuous coating or as a printed (intermittent) pattern.

The thickness of the adhesive drug-containing layer (drug reservoir layer) and optional skin contact layers of the compositions of this invention can vary, depending upon such factors as the amount of drug to be delivered from the composition and the desired wear period.

Although such processes can be used for any sized patch, it is particularly suited for use in patches having a surface area ranging from between about 2 cm² and about 15 cm², and preferably ranging from between about 5 cm² and about 10 cm², e.g., a patch having a surface area of about 5 cm² and a patch having a surface area of about 10 cm².

In various embodiments of the disclosure, the patches are of any suitable thickness, e.g., about 20 microns or greater in total thickness, including the release liner layer. Certain embodiments of the disclosure range from between about 20 microns to about 1,500 microns in total thickness. In some embodiments, the patches have a total thickness of about 250 microns.

Furthermore, the present invention allows for the processing of high viscosity adhesive layers or those containing thermally labile and/or highly volatile APIs.

The present disclosure allows for fine tuning or adjustment of the delivery rates of the API by varying the mechanics of the process used to make the adhesive layer or the properties/arrangements of the layers in the resulting device. For example, one skilled in the art will be able to vary drug loading in the adhesive layer, the thickness of the adhesive layer, the inclusion of additional layers, such as membrane layers, so as to provide optimal API delivery rates. Moreover, one skilled in the art could manipulate parameters of the extrusion process, if used, including the size and shape of the augers/screws used, the speed of extrusion, and temperatures utilized during processing to make adhesive layers having different properties.

In certain embodiments of the present disclosure, the thickness of the pharmaceutical composition layer in the transdermal device of the present disclosure ranges from about 20 to about 1000 microns, more preferably from about 60 to about 100 microns.

In various embodiments of the present disclosure, the transdermal device is formed as a continuous sheet or web and may be cut into desired width and length, or separated along a frangible area dividing each device, into patches before use although such devices may be provided as discrete patches which are cut with a die into the desired shape, e.g., circular.

The transdermal devices of the invention in general have, for example an effective contact area of pharmaceutical composition on the skin of from about 1 to about 80 cm², preferably about 10 cm², and are intended to be applied at intervals of about once every 1 to 7 days, preferably 1-3 days. Active pharmaceutical ingredient or drug rivastigmine is well tolerated at a potency of 36 mg in free base form in patches of up to 80 cm² contact area, according to the invention. Typically, 12 mg doses of rivastigmine can be absorbed from such a patch. Rivastigmine may, for example, be administered at a dose of about 4.6 mg in a patch of about 5 cm², twice every day, or at a dose of about 9.5 mg in a patch of about 10 cm², once every twenty-four hours. The patch may be applied, for example on the abdomen, thigh, behind an ear, or on a shoulder or upper arm.

Pharmaceutical compositions, formed as a transdermal device, of the present disclosure are useful for the same indications as for known compositions containing active pharmaceutical ingredient or drug. The exact amounts of active pharmaceutical ingredient to be administered may depend on a number of factors, e.g. the drug release characteristics of the compositions, the drug penetration rate observed in vitro and in vivo, the duration of action required, the form of active pharmaceutical ingredient, and for transdermal compositions the size of the skin contact area, and the part of the body to which the unit is fixed. The amount of active pharmaceutical ingredient and surface area of the patch may be optimized through routine bioavailability tests measuring the blood levels of active agents after administration of the active pharmaceutical ingredient composition to intact skin and comparing those blood levels to those following oral administration of a therapeutically effective dose of the active pharmaceutical ingredient.

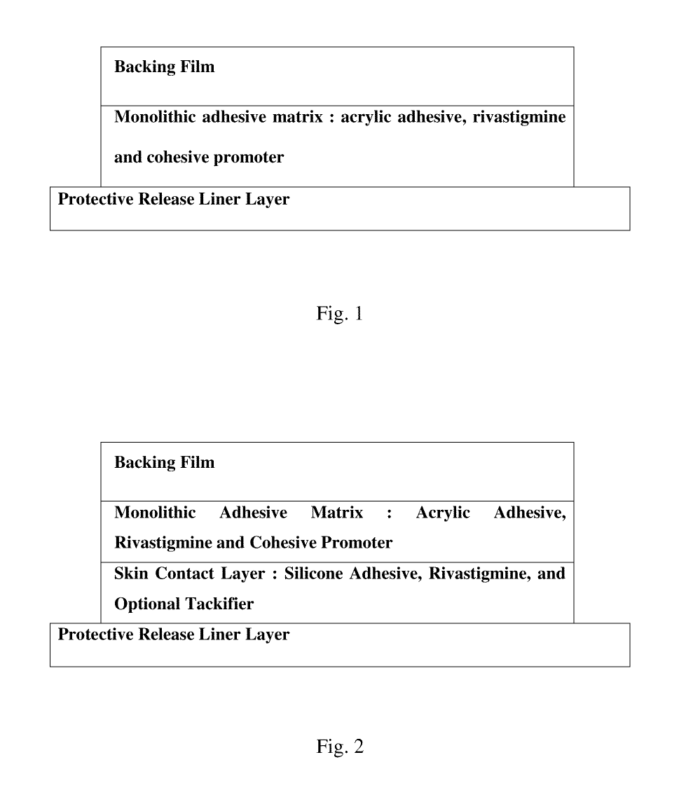

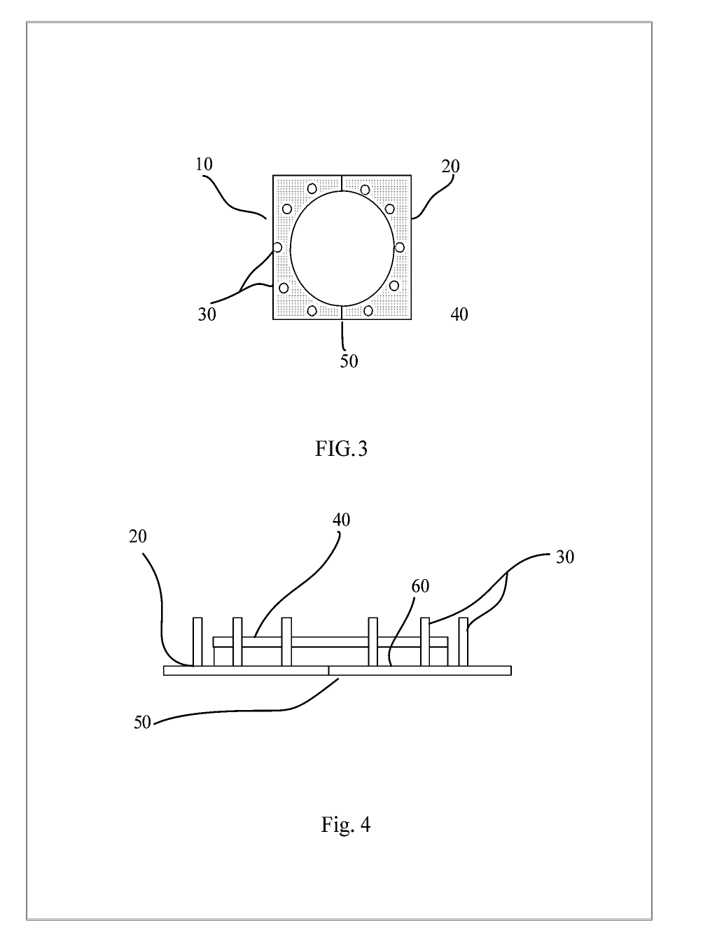

With reference to the FIGURES, the transdermal device of the present disclosure can be further explained as follows:
FIG. 1 depicts a schematic diagram of the product of the present disclosure showing i) a backing film as the upper layer, ii) an intermediate layer comprising a monolithic adhesive matrix which comprises a) acrylic adhesive, b) active pharmaceutical ingredient, e.g., rivastigmine and c) cohesive promoter, and iii) a lower layer comprising a protective release liner which is slit or scored to permit easy removal of the liner, in two parts, from the rest of the product.
FIG. 2 depicts a schematic diagram of the product of the present disclosure showing a backing film as the upper layer, an intermediate layer comprising a) a multiple drug reservoir layer, i.e., a bilayer comprising A) an upper acrylic drug reservoir matrix layer comprising i) acrylic adhesive, ii) active pharmaceutical ingredient, e.g., rivastigmine, and iii) cohesive promoter; and B) a lower skin contact layer comprising i) silicone adhesive, ii) active pharmaceutical ingredient, e.g., rivastigmine, and iii) tackifier, and a lower layer comprising the protective release liner which is slit or scored to permit easy removal of the liner prior to use.
FIG. 3 depicts an overhead view of an embodiment of the transdermal drug delivery device 10 of the present disclosure. The device comprises a rectangular release liner layer 20 which is stamped from the bottom to form protective projections 30 around the perimeter of the circular patch affixed to the release liner layer. The projections, which can range from about 1 to about 10 mm, e.g., from about 2 to about 6 mm in length, and from about 0.2 to 3 mm, e.g., from about 1 to 3 mm in diameter, serve to prevent extensive contact of the exterior surface of the device with the interior surface of the envelope or pouch in which it is stored (not shown). The circular patch comprises a skin-colored backing layer 40 and one or more intermediate layers (not shown) comprising the drug and/or skin adhesive. The bottom surface of the intermediate layer is releasably attached to the release liner layer which further comprises a slit 50 at or near its midline to assist the user in removal of the release liner layer before use.
FIG. 4 depicts a side view of an embodiment of the transdermal drug delivery device 10 of the present disclosure. Shown in profile, the device comprises a square release liner layer 20 which is stamped with a die from the bottom to form protective projections 30 around the perimeter of the circular patch affixed to the release liner layer. The projections, which extend above the surface of the backing layer 40, prevent extensive contact of the exterior surface of the backing layer with the interior surface of the envelope or pouch in which it is stored (not shown). The circular patch comprises a skin-colored backing layer 40 and intermediate layer 60 comprising the drug and skin adhesive. The intermediate layer is releasably attached to the release liner layer 20 which further comprises a slit 50 at or near its midline to assist the user in removal of the release liner layer before use.

The invention will now be described in more detail by the following non-limiting EXAMPLES. In all the EXAMPLES, no antioxidant is added to the polymer matrix containing the active pharmaceutical ingredient (S)—N-ethyl-3-[(I-dimethylamino)ethyl]-N-methylphenylcarbamate. The EXAMPLES are presented to illustrate but a few embodiments of the invention. All parts are by weight unless otherwise indicated.

### Example 1

### Comparative—Unpouched Storage

A bi-layer adhesive matrix was prepared which comprised a drug reservoir matrix layer containing i) (S)—N-ethyl-3-[(1-dimethylamino)ethyl]-N-methylphenyl carbamate, i.e., rivastigmine, ii) ethyl cellulose as cohesive promoter and iii) silicone adhesive (Dow Corning® BIO-PSA 7-4202). A skin contact adhesive layer was also provided which contained a mixture of BIO-PSA 7-4202 and BIO-PSA 7-4302-two amine-compatible silicone adhesives which are available from Dow Corning. The bi-layer adhesive matrix was positioned between a protective release liner of Scotchpak™ 1022 available from 3M and a backing film MEDIFLEX® 1501, available from Mylan Technologies. Patches were die cut from the resulting laminate with a circular die. The ingredients in the bi-layer adhesive matrix in each 5 cm² patch are listed in TABLE 1 below. The two primary oxidation impurities of the drug substance were measured at 1.07 wt. % and 1.00 wt. %, respectively, relative to rivastigmine content after storage of the unpouched patches at 105° C. for 65 hours.

TABLE 1
Patch Composition of Example 1 (Unpouched Components)

| | Wt. (mg per |
|---|---|
| Components | unit patch |
| Drug Reservoir Layer: | |
| (S)-N-ethyl-3-[(1-dimethylamino)ethyl]-N-methylphenyl | 9.0 |
| carbamate | |
| Ethyl Cellulose | 6.0 |
| Silicone Adhesive BIO-PSA 7-4202 | 15.0 |
| Skin Contact Layer | |
| Silicone Adhesive BIO-PSA 7-4202 | 7.5 |
| Silicone Adhesive BIO-PSA 7-4302 | 7.5 |

### Example 2

Patches die cut from the bi-layer laminate described in EXAMPLE 1 were pouched and heat sealed with AP982 pouch stock (35# C1S paper (coated on one side)/7.5# LDPE/0.00035″ foil/N-481 adhesive/0.03175 mm (1.25 mil) Barex® 210). AP982 pouch stock is available from American Packaging Corporation of Rochester, N.Y., USA. Barex® Film Grade resin, available from INEOS Barex of Newark, Del., USA, is an impact modified acrylonitrile-methyl acrylate copolymer with excellent gas barrier properties and a wide range of chemical resistances.

The two primary oxidation impurities of the drug substance were measured as 0.92 wt. % and 0.81 wt. %, respectively, based on total drug content after storage of the pouched patches at 105° C. for 65 hours. The impurity levels of the pouched patches in EXAMPLE 2 were lower than the impurity levels of the unpouched patches of EXAMPLE 1.

### Example 3

Patches were prepared using a bi-layer system whose drug reservoir adhesive matrix layer contained (S)—N-ethyl-3-[(1-dimethylamino)ethyl]-N-methylphenyl carbamate, ethyl cellulose (as cohesive promoter), and silicone adhesive (Bio-PSA 7-4202). A skin contact adhesive layer was also provided which comprised a mixture of BIO-PSA 7-4202 and BIO-PSA 7-4302-two amine-compatible pressure sensitive silicone adhesives available from Dow Corning. The bi-layer laminate was positioned between a protective release liner of Scotchpak™ 9744 available from 3M and a backing film of MEDIFLEX® 1501. The resulting four layer laminate was die cut to form patches of five cm². Ingredients in the bi-layer adhesive matrix for each patch are listed in TABLE 2 below.

Each patch was placed and heat sealed in a separate four layer laminate pouch, made from AP723 pouch stock (26# CIS paper/7.2# LDPE/0.00035″ foil/14.4# LDPE). AP723 pouch stock is available from American Packaging Corporation.

The pouches were stored for three months in a stability chamber at 40° C. at 75% relative humidity. The two primary oxidation impurities of the drug rivastigmine were measured and found to be 0.13%, and 0.19% relative to rivastigmine content after three months.

TABLE 2
Patch Composition of Example 3 (Pouched with AP723)

| | Wt. (mg per |
|---|---|
| Components | unit patch |
| Drug Reservoir Layer: | |
| (S)-N-ethyl-3-[(1-dimethylamino)ethyl]-N-methylphenyl | 9.0 |
| carbamate | |
| Ethyl Cellulose | 6.0 |
| Silicone Adhesive BIO-PSA 7-4202 | 15.0 |
| Skin Contact Layer | |
| Silicone Adhesive BIO-PSA 7-4202 | 7.5 |
| Silicone Adhesive BIO-PSA 7-4302 | 7.5 |

### Example 4

Patch compositions were prepared in accordance with TABLE 3 below. The adhesive matrix was a bi-layer system. The drug reservoir matrix layer contained (S)—N-ethyl-3-[(1-dimethylamino)ethyl]-N-methylphenyl carbamate, ethyl cellulose (as cohesive promoter) and silicone adhesive (BIO-PSA 7-4202). The skin contact adhesive layer was made solely of the silicone adhesive BIO-PSA 7-4202, an amine-compatible pressure sensitive silicone adhesive available from Dow Corning.

Each patch was placed and heat sealed in a separate four layer laminate pouch, AP982, further disclosed in EXAMPLE 2 above.

The pouches were stored for three months in a stability chamber at 40° C. at 75% relative humidity. The two primary oxidation impurities of the drug rivastigmine were measured and found initially to be 0.06% and 0.09%, based on total drug content. Those levels increased to 0.13%, and 0.23%, respectively, based on total drug content after three months' storage.

TABLE 3
Patch Composition of Example 3 (Pouched with AP982)

| | Wt. (mg per |
|---|---|
| Components | unit patch |
| Drug Reservoir Layer: | |
| (S)-N-ethyl-3-[1-dimethylamino)ethyl]-N-methylphenyl | 9.0 |
| carbamate | |
| Ethyl Cellulose | 6.0 |
| Silicone Adhesive BIO-PSA 7-4202 | 15.0 |
| Skin Contact Layer | |
| Silicone Adhesive BIO-PSA 7-4202 | 7.5 |

### Example 5

The adhesive matrix was a bi-layer system whose drug reservoir matrix layer contained (S)—N-ethyl-3-[(1-dimethylamino)ethyl]-N-methylphenyl carbamate, ethyl cellulose (as cohesive promoter) and acrylic adhesive (Duro-Tak™ 87-2353). A skin contact adhesive layer was also provided of silicone adhesive BIO-PSA 7-4202, a standard pressure sensitive silicone adhesive available from Dow Corning.

The bi-layer laminate was manufactured between a protective release liner of Scotchpak™ 9744 available from 3M and a backing film MEDIFLEX® 1501. The resulting four layer laminate was die cut to form 5 cm² patches. The ingredients in the bi-layer adhesive matrix for each patch are listed in TABLE 4 below.

Each patch was placed and heat sealed in a separate child-resistant four layer laminate pouch, made from AP1318 pouch stock (92 ga PET/N-177/7.5# WLDPE/0.00035″Foil/N-481 Adhesive/0.03175 mm (1.25 mil) Barex® film). AP1318 pouch stock is available from American Packaging Corporation.

The pouches were stored for three months in a stability chamber at 40° C. at 75% relative humidity. The two primary oxidation impurities of the drug rivastigmine were measured and found to be 0.04 wt. % and 0.03 wt. %, initially. Those levels increased to 0.14 wt. %, and 0.16 wt. %, respectively, based on total drug content after three months' storage.

The total impurity of the drug substance was found to have increased from 0.20% at time zero to 0.40% after three months.

TABLE 4
Patch Composition of Example 4 (Pouched with AP982)

| | Wt. (mg per |
|---|---|
| Components | unit patch |
| Drug Reservoir Layer: | |
| (S)-N-ethyl-3-[(1-dimethylamino)ethyl]-N-methylphenyl | 9.0 |
| carbamate | |
| Ethyl Cellulose | 6.0 |
| Acrylate Adhesive Duro-Tak ™ 87-2353 | 15.0 |
| Skin Contact Layer | |
| Silicone Adhesive BIO-PSA 7-4202 | 15.0 |

### Example 6

EXAMPLE 4 was repeated except that during the packaging process the headspace of each pouch was flushed with ultrapure nitrogen gas for 3 seconds before the pouch enclosing the patch was heat sealed, providing an environment surrounding the stored patches of no greater than 5 wt. % oxygen. The patches were then stored at 40° C. at 75% relative humidity. The two primary oxidation impurities of the drug rivastigmine were measured and initially found to be 0.06% and 0.09%, initially relative to total drug content. Those levels increased to 0.09%, and 0.15%, respectively, after three months' storage which is lower than the impurity level in EXAMPLE 4 in which the pouch was not purged with an inert gas.

### Example 7

Example 5 was again repeated except that during the packaging process an oxygen scavenger packet was enclosed within the child resistant pouch made of AP1318 multilaminate prior to heat sealing.

The pouched patches were then stored at 40° C. at 75% relative humidity for three months. The two primary oxidation impurities of the drug rivastigmine were measured and initially found to be 0.04 wt % and 0.03 wt %. These oxidation impurity levels remained the same even when measured after three months' storage. Moreover, the total impurity of the drug substance after three months did not increase from the initially measured level of 0.20 wt %, even after three months of storage.

### Example 8

A patch was prepared having a bi-layer system for its adhesive matrix which comprised a) drug reservoir matrix layer containing (S)—N-ethyl-3-[(1 dimethylamino)ethyl]-N-methylphenyl carbamate (i.e., rivastigmine), Plastoid® B as cohesion promoter and Acrylate adhesive Duro-Tak™ 87-235A and b) a skin contact layer of silicone adhesive (BIO-PSA 7-4202), an amine-compatible pressure sensitive silicone adhesive available from Dow Corning. Further details are set out in TABLE 4 above.

The resulting patch was packaged in a child resistant pouch, AP1318, which was the same pouch material described in EXAMPLE 5 above. For some patch samples, pieces of Scotchpak™ 9744 release liner were used to sandwich the patch during the packaging process. Once the patch was sandwiched, an oxygen scavenger packet, as described in EXAMPLE 7 was introduced into the pouch and attached to an internal wall thereof. The pouch was then heat sealed and the sealed patches were stored at 40° C., 75% relative humidity for three months. Drug substance adsorption into the oxygen scavenger packet was measured for both sandwiched and unsandwiched samples.

After 3 months storage at 40° C., 75% relative humidity, drug absorption was measured at 9.6 wt. % in the absence of overlay and underlay, i.e., for the unsandwiched samples. In contrast, the absorption level was reduced to between 2.7 wt. % and 2.9 wt. % in the presence of overlay and underlay, i.e., for the sandwiched samples.

These results show that use of impermeable overlay and underlay films to sandwich a patch does not impair the capability of the oxygen scavenging packet as shown by the impurity levels measured. In those instances where an oxygen scavenger packet was used, the levels of the two primary oxidation impurities of the drug substance were initially measured as 0.06 wt. % and 0.03 wt. % of the drug substance. Those levels increased to 0.11 wt. % and 0.10 wt. % after the three months' storage. When an oxygen scavenger packet was used, the levels of the two primary oxidation impurities were initially measured as 0.06 wt. % and 0.03 wt. %. After three months' storage those levels only slightly increased to 0.06 wt. % and 0.06 wt. %, respectively. The total impurity level of the active ingredients increased only slightly from 0.21% at time zero to 0.23% after three months.

### Example 9

A bi-layer adhesive matrix system was prepared whose drug reservoir matrix layer contained (S)—N-ethyl-3-[(1-dimethylamino)ethyl]-N-methylphenyl carbamate as the drug (API), Plastoid® B as cohesion promoter and Duro-Tak™ 87-235A as adhesive. A skin contact adhesive layer was also provided which was a mixture of Dow Corning® BIO-PSA 7-4202 amine-compatible pressure sensitive silicone adhesive and Medical Fluid 360, a Dow Corning silicone oil added as a tackifier. Additional details are set out in TABLE 5 below.

The bi-layer laminate was placed between a protective release liner of Scotchpak™ 9744 available from 3M and a backing film MEDIFLEX® 1501. The resulting four layer laminate was die cut to form 5 cm² patches. The ingredients in the bi-layer adhesive matrix for each patch are listed in TABLE 5 below.

Each patch was placed and heat sealed in a separate four layer laminate pouch, made from AP1318 pouch stock available from American Packaging Corporation (92 ga PET/N-177/7.5# WLDPE/0.00035″ Foil/N-481 Adhesive/0.03175 mm (1.25 mil) Barex® film).

The release liner layer is oversized relative to the rest of the patch for ease of removal when the patch is to be used. The release liner layer is stamped to provide small indentations on the outside which provide projections on the inside surface facing the patch. The projections assist in protecting the backing layer from directly contacting the inside surface of the pouch during storage and handling. FIG. 3 and FIG. 4 respectively provide overhead and side views of the patch and show the relative location and sizes of the respective patch layers, as well as the location of the slit in the release liner layer which facilitates its removal by the user. The patch and release underlay are packaged into a child-resistant Barex® pouch, under a nitrogen-purged environment with an oxygen level no greater than about 5 wt. %.

TABLE 5
Patch Composition of Example 10 (Pouched with AP1318)

| | Wt. (mg per | |
|---|---|---|
| Components | unit patch | % w/w |
| Backing | | |
| Pigmented Polyethylene/PET Backing- | | |
| (MEDIFLEX ® 1501) | | |
| Drug Reservoir Layer: | | |
| (S)-N-ethyl-3-[(1-dimethylamino)ethyl]-N- | 18.00 | 30.0 |
| methylphenyl carbamate (Rivastigmine) | | |
| Plastoid ® B | 12.00 | 20.0 |
| Acrylate Adhesive Duro-Tak ™® 87-235A | 30.00 | 50.0 |
| Total: | 60.00 | 100.0 |
| Skin Contact Layer | | |
| Medical Fluid 360 (Silicone Oil Tackifier) | 0.80 | 2.0 |
| Silicone Adhesive BIO-PSA 7-4202 | 39.20 | 98.0 |
| Total: | 40.0 | 100.0 |
| Release Liner | | |
| Fluoropolymer-Coated Polyester Release Liner | | |
| (Scotchpak ™ 9744) | | |
| Total Finished Adhesive Matrix Weight: | 100.0 | |

Although various exemplary embodiments have been described in detail with particular reference to certain exemplary aspects thereof, it should be understood that the subject matter disclosed herein is capable of other different embodiments, and its details are capable of modifications in various obvious respects. As is readily apparent to those skilled in the art, variations and modifications can be affected while remaining within the spirit and scope of the disclosure. Accordingly, the foregoing disclosure, description, and figures are for illustrative purposes only, and do not in any way limit the invention, which is defined only by the claims.

What is claimed is:

1. A transdermal drug delivery device comprising:
a) a substantially impermeable backing layer;
b) an adhesive drug-containing layer substantially free of antioxidant and containing a therapeutically effective amount of an oxidizably and/or hydrolyzably degradable drug, which adhesive drug-containing layer is capable of adhering directly to a subject's skin or indirectly via one or more optional intermediate layers at least one of which is another adhesive layer capable of adhering to a subject's skin; and
c) a substantially impermeable protective release liner layer which releasably contacts the adhesive drug-containing layer or the another adhesive layer;
wherein the delivery device is sealed within a substantially oxygen impermeable degradation protective packaging system.

2. The drug delivery device of claim 1 wherein the packaging system comprises a degradation protectant.

3. The drug delivery device of claim 2 wherein the degradation protectant is selected from the group consisting of an inert gas, an antioxidant, an oxygen scavenger, a moisture scavenger, and a combination thereof.

4. The drug delivery device of claim 1 wherein the packaging system is filled with a substantially inert gas and the oxygen level in the packaging system is no greater than about 5 wt. %.

5. The drug delivery device of claim 4 wherein the inert gas is nitrogen.

6. The drug delivery device of claim 1 wherein the drug comprises an acetylcholinesterase inhibitor.

7. The drug delivery device of claim 1 wherein the drug comprises a phenyl carbamate.

8. The drug delivery device of claim 1 wherein the drug is rivastigmine in the form of a free base or acid addition salt.

9. The drug delivery device of claim 1 wherein the drug is in a solid form.

10. The drug delivery device of claim 1 wherein the drug is in a liquid form.

11. The drug delivery device of claim 1 wherein
a) the substantially impermeable backing layer contains at least one of polyethylene terephthalate, nylon, polyethylene, polypropylene, polyester, polyester/ethylene-vinyl acetate, metallized polyester film, polyvinylidene chloride, metal foil, polyvinylidene fluoride film, ethylene vinyl acetate film laminated to a polyester, and ethylene vinyl acetate film laminated to a metallized polyester;
b) the adhesive drug-containing layer contains
i) at least one acrylic adhesive which is selected from acrylate co-polymer and cross-linked acrylate copolymer;
ii) a cohesive promoter which is selected from polymers of methacrylate containing alkyl (C₁₋₄) ester groups, polymers of methacrylate esters containing trimethylaminoethyl cationic ester groups and other neutral (C₁₋₄) alkyl ester groups, a mixture of an acrylate polymer and a methacrylate polymer, polymers of acrylate esters containing methyl and ethyl neutral ester groups and trimethylaminoethyl cationic ester groups,
iii) optionally, at least one intermediate layer comprising 1) at least one skin contact layer adhesive selected from silicone, natural rubber, synthetic rubber, polyisobutylene, neoprene, polybutadiene, polyisoprene, polysiloxane, cross-linked acrylic copolymer, uncross-linked acrylic copolymer, vinyl acetate adhesive, polyacrylate, ethylene vinyl acetate copolymer, styrene-isoprene copolymer, polyurethane, plasticized polyether block amide copolymer, plasticized styrene-rubber block copolymer, and mixtures thereof; 2) an optional drug component, and 3) an optional silicone oil tackifier of appropriate molecular weight; and
iv) optionally, at least one intermediate layer comprising a membrane layer made of a flexible, polymeric material selected from low density polyethylene, high density polyethylene, ethylene vinyl acetate copolymers, and polypropylene; and
c) the substantially impermeable protective release liner layer has a moisture vapor transmission rate (MVTR) of less than 20 g/m²·24 hr and comprises at least one of polyethylene terephthalate/silicone, polyethylene terephthalate/aluminized polyester coated with silicone, polyester with a silicone coating, polyurethane with a silicone coating, polyester with a fluorocarbon coating, polyurethane with a fluorocarbon coating, polyester with a fluorosilicone coating, polyurethane with a fluorosilicone coating, polyolefin coated with a fluoropolymer release agent, polyester coated with a fluoropolymer release agent, paper, thermoplastics, polyester film, and metal foil; and
the substantially oxygen impermeable degradation protective packaging system has an oxygen transmission rate of less than about 0.05 ml/100 in²/24 hr/bar measured at 22° C. (72° F.), and comprising a sealable thermoplastic pouch containing an acrylonitrile-methyl acrylate copolymer, with the packaging system further comprising a degradation protectant selected from at least one of an inert gas, an antioxidant, an oxygen scavenger, and a moisture scavenger.

12. The drug delivery device of claim 1 wherein
a) the substantially impermeable backing layer is a film containing at least one layer selected from polyethylene terephthalate, nylon, polyethylene, polypropylene, polyester, polyester/ethylene-vinyl acetate, and metallized polyester;
b) the adhesive drug-containing layer contains
i) at least one acrylic adhesive which is selected from a) uncross-linked copolymer comprising a first monomer selected from butyl acrylate, ethyl hexyl acrylate and vinyl acetate, and a second monomer different than the first monomer; and b) cross-linked copolymer comprising a third monomer selected from butyl acrylate, ethyl hexyl acrylate and vinyl acetate, and a fourth monomer different than the third monomer;
ii) a cohesive promoter which is selected from polymers of methacrylate containing alkyl (C₁₋₄) ester groups, polymers of methacrylate esters containing trimethylaminoethyl cationic ester groups and other neutral (C₁₋₄) alkyl ester groups, and a mixture of an acrylate polymer and a methacrylate polymer;
iii) optionally, at least one intermediate layer comprising 1) at least one skin contact layer adhesive selected from silicone, natural rubber, synthetic rubber, polyisobutylene, neoprene, polybutadiene, polyisoprene, polysiloxane, cross-linked acrylic copolymer, and uncross-linked acrylic copolymer; 2) an optional drug component, and 3) an optional silicone oil tackifier of appropriate molecular weight;
iv) optionally, at least one intermediate layer comprising a membrane layer made of a flexible, polymeric material selected from low density polyethylene, high density polyethylene, ethylene vinyl acetate copolymers, and polypropylene;
c) the substantially impermeable protective release liner layer having a moisture vapor transmission rate (MVTR) of less than about 15 g/m²·24 hr and comprises at least one of polyester film coated with a fluoropolymer release agent and polypropylene film coated with a fluoropolymer release agent; and
the substantially oxygen impermeable degradation protective packaging system has an oxygen transmission rate of less than about 0.03 ml/100 in²/24 hr/bar measured at 22° C. (72° F.), and comprising a sealable pouch containing an oxygen impermeable, acrylonitrile-methyl acrylate copolymer film, the packaging system further comprising a degradation protectant selected from at least one of an inert gas, an antioxidant, an oxygen scavenger, and a moisture scavenger.

13. The drug delivery device of claim 1 wherein
a) the substantially impermeable backing layer comprises a three layer structure of polyethylene/polyurethane adhesive/polyethylene terephthalate;
b) the adhesive drug-containing layer contains i) rivastigmine, ii) acrylate copolymer cohesive promoter, and iii) pressure sensitive adhesive comprising a copolymer of butyl acrylate, ethyl hexyl acrylate and vinyl acetate and the another adhesive layer capable of adhering to a patient's skin comprises a silicone oil tackifier and an amine compatible silicone adhesive;
c) the substantially impermeable protective release liner layer contains a fluoropolymer coated polyester film; and
d) the substantially oxygen impermeable degradation protective packaging system comprises a sealable multilayer pouch comprising, from its external surface, polyester film/adhesive/polyethylene film/aluminum foil/adhesive/heat-sealable, oxygen impermeable, acrylonitrile-methyl acrylate copolymer film, the packaging system further comprising a degradation protectant comprising nitrogen with an oxygen level in the pouch of no greater than about 5 wt. %.

14. The drug delivery device of claim 1 wherein the degradation protective packaging system comprises a sealable plastic layer which is sealable by at least one of heat, pressure, solvent, and adhesive.

15. The drug delivery device of claim 1 wherein the degradation protective packaging system comprises a heat sealable plastic layer.

16. The drug delivery device of claim 1 wherein the adhesive drug-containing layer contains antioxidant at levels insufficient to stabilize the drug against degradation from oxidation and/or hydrolysis.

17. The drug delivery device of claim 1 wherein each substantially impermeable layer comprises a moisture vapor transmission rate of less than 20 g/m²·24 hr.

18. The drug delivery device of claim 1 wherein each substantially impermeable layer comprises a moisture vapor transmission rate of from about 1 g/m²·24 hr to about 15 g/m²·24 hr.

19. The drug delivery device of claim 1 wherein the substantially oxygen impermeable degradation protective packaging system comprises a substrate containing an antioxidant, separate from the drug delivery device.

20. A method for preventing degradation of a transdermal drug delivery device of the type comprising a drug reservoir positioned between a backing layer and a release liner layer comprising: (a) providing the transdermal drug delivery device with a substantially impermeable backing layer and a substantially impermeable release liner each having a moisture vapor transmission rate of less than 20 g/m²·24 hr; (b) providing a degradation protectant within a substantially oxygen impermeable pouch or pouch precursor; (c) placing the device within the pouch or pouch precursor; (d) optionally placing an antioxidant-containing substrate within the pouch or pouch precursor; and (e) sealing the pouch or pouch precursor.

21. The method of claim 20 wherein the drug reservoir comprises rivastigmine in the form of a free base or acid addition salt.

22. A method for preparing a transdermal drug delivery device of the type which is resistant to degradation of the drug during storage comprising:
i) attaching a substantially impermeable backing layer to one side of an adhesive drug-containing layer substantially free of antioxidant and containing a therapeutically effective amount of an oxidizably and/or hydrolyzably degradable drug, which adhesive drug-containing layer is capable of adhering directly to a patient's skin or indirectly via one or more optional intermediate layers at least one of which is another adhesive layer capable of adhering to a patient's skin;
ii) attaching to the other side of the adhesive drug-containing layer, or the optional another adhesive layer if present, a substantially impermeable protective release liner layer which releasably contacts the adhesive drug-containing layer or the another adhesive layer; and
iii) sealing the product of steps i) and ii) within a substantially oxygen impermeable degradation protective packaging system.

23. The method of claim 22 wherein the drug comprises rivastigmine in the form of a free base or acid addition salt.

24. A method for preparing a transdermal drug delivery device of the type which is resistant to degradation of the drug during storage comprising:
i) coating a first release liner with a liquid precursor to a solid matrix reservoir layer containing an oxidizably and/or hydrolyzably degradable drug and substantially free of antioxidant;
ii) drying the liquid precursor to provide a solid matrix reservoir layer-coated first release liner;
iii) laminating the coated side of the solid matrix reservoir layer-coated first release liner to a substantially impermeable backing layer;
iv) removing the first release liner to provide an exposed solid matrix reservoir layer surface;
v) coating a second release liner with a liquid precursor to a solid adhesive skin contact layer;
vi) drying the liquid precursor of step (v) to provide a solid adhesive skin contact layer-coated release liner having an exposed solid adhesive skin contact layer surface;
vii) laminating the exposed solid adhesive skin contact layer surface of step vi) to the exposed solid matrix reservoir layer surface of step iv) to provide a multi-laminate comprising from its outside to inside a) an external backing layer, b) a solid matrix reservoir, c) a solid adhesive skin contact layer, and d) a release liner layer;
viii) slitting and/or die cutting the multi-laminate to provide an individual patch of desired width and/or shape; and
ix) individually sealing the patch within a substantially oxygen impermeable degradation protective packaging system.
